(19) United States
(12) Patent Application Publication
(10) Pub. No.: US 2016/0287750 A1
(43) Pub. Date: Oct. 6, 2016

(54) STANDARDIZED AMNIOTIC MEMBRANE TISSUE GRAFT

(71) Applicant: Applied Biologics, LLC, Scottsdale, AZ (US)

(72) Inventor: Edward Britt, Scottsdale, AZ (US)

(21) Appl. No.: 14/986,182

(22) Filed: Dec. 31, 2015

Related U.S. Application Data
(60) Provisional application No. 62/098,992, filed on Dec. 31, 2014.

(51) Int. Cl. A61L 27/36

(52) U.S. Cl. CPC A61L 27/3633; A61L 2300/00

(57) ABSTRACT

The invention relates to preparations of amniotic membrane-derived tissue grafts standardized for the concentration amniotic membrane and its constituent components by dry weight. The standardized amniotic membrane tissue graft is used by physicians and other healthcare providers in the surgical and minimally invasive medical therapy of a wide range of injuries and disease processes. The preparations maximize available quantities of non-cellular bioactive compounds and viscosity to enhance therapeutic efficacy for the desired application. The standardized amniotic membrane tissue graft preparations are semi-viscous fluids or gels which may be intraoperatively transplanted at the recipient site using a needless syringe, by non-operative percutaneous injection through a hypodermic needle, or by direct application.

### CROSS REFERENCE TO RELATED APPLICATION

This application claims the benefit of U.S. Provisional Application No. 62/098,992, filed Dec. 31, 2014, the contents of which are incorporated entirely herein by reference.

### BACKGROUND OF THE INVENTION

1. Technical Field

This invention relates to the preparation of an amniotic membrane tissue graft using amnion and amniotic fluid; in particular, embodiments of the invention relate to the preparation and use of a standardized mammalian tissue graft product formed by reconstituting a measured, standardized quantity of dried, ground amniotic membrane with a fluid.

2. State of the Art

Amniotic membrane, specifically human amniotic membrane, has been used as a therapeutic agent for over one hundred years. The amnion's interstitial matrix and cellular components contain a complex biologic soup of growth factors, inflammatory mediators, immuno-modulators, and other active biomolecules. Amniotic membrane is rich in embryonic stem cells containing high concentrations of these biologically active substances.

Amniotic membrane is used in a variety of surgical procedures as an adjunct to healing and to minimize formation of scar tissue and adhesions. The amniotic membrane is typically dried prior to packaging, sterilization, and storage. Some preparations, however, reconstitute the dried amniotic membrane using a tissue preservative solution prior to the packaging and sterilization for storage. In some preparations, the amniotic membrane is ground in a tissue mill prior to drying such that the resulting reconstituted amniotic membrane product is a fluid. The medium used to reconstitute the dried amniotic membrane is typically an isotonic solution containing water and electrolytes, but no growth factors, other active biomolecules, or additional extraembryonic stem cells. The reconstituted amnion varies in viscosity and concentration of physiologically active small and large biomolecules. Consequently, it may be difficult for clinicians and impossible for researchers to obtain reproducible results with the use of a non-standardized product.

Accordingly, what is needed is a reconstituted dried amniotic membrane tissue graft in a standardized preparation for clinical and investigational use.

Citation of documents herein is not an admission by the applicant that any is pertinent prior art. Stated dates or representation of the contents of any document is based on the information available to the applicant and does not constitute any admission of the correctness of the dates or contents of any document.

### DISCLOSURE OF EMBODIMENTS OF THE INVENTION

It will be appreciated by practitioners in the art that certain tissue graft properties are more desirable or well-suited for various applications. For instance, properties such as viscosity, particle size, and particle concentration can affect how well a particular tissue graft is suited to administration at a soft tissue or hard tissue site on a patient in need of a tissue graft. Furthermore, practitioners in the art will appreciate that the lack of available tissue grafts with known standardized properties presents a tremendous disadvantage in terms of consistently identifying and obtaining tissue grafts suited for a particular application. This is especially true in the case of tissue grafts that include amniotic membrane particles as a major component, due to the variability in available amniotic membrane tissue properties and the resulting variability in tissue grafts produced from such components.

The advantage of having an available source of standardized tissue grafts with known properties is the consistency that a tissue graft with such properties provides to the practitioner and the subject in need of the tissue graft. For example, one can select a tissue graft or a set of tissue grafts from a source with the knowledge that properties such as viscosity, amniotic membrane particle size, and/or amniotic membrane particle concentration are consistent between individual tissue grafts. This presents an advantage to a practitioner and/or subject in need of a tissue graft or multiple tissue grafts with specific properties. The ability to obtain tissue grafts with standardized properties is particularly advantageous in instances where the tissue graft is selected for application to a subject at a particular location. For instance, a tissue graft with particular amniotic membrane particle concentration, amniotic membrane particle size, and viscosity values may be better suited for application to a hard tissue (i.e., bone) site than a soft tissue (e.g., skin or muscle) site in or on a patient. The availability of a source of standardized tissue grafts with particular known properties would be highly advantageous in such a situation since it would allow one to consistently procure one or more tissue grafts with optimal properties for application to a particular site in or on a subject.

For instance, in general, in the case of application of an amniotic membrane tissue graft to soft tissue, it is advantageous to use tissue grafts with a relatively low concentration of amniotic membrane particles but with high viscosity and relatively large amniotic membrane particles. On the other hand, in general, in the case of application of an amniotic membrane tissue graft to hard tissue such as bone, it is advantageous to use tissue grafts with a relatively high concentration of amniotic membrane particles but with low viscosity and smaller amniotic membrane particles. In some instances, it may be advantageous to include hydroxyapatite in tissue grafts intended for application to bone. However, at present, no such standardized tissue grafts with particular properties suited to particular applications or methods of applying such tissue grafts is known in the art.

The current invention provides a solution to these problems by providing standardized tissue grafts and sets of standardized tissue grafts that include a fluid and a standardized quantity of dried amniotic membrane particles with standardized properties. The standardized properties in tissue grafts of the invention will be advantageous to performing specific procedures on a subject, for instance applying tissue grafts to bone or soft tissue in or on a subject, because the practitioner will be able to knowingly obtain one or multiple tissue grafts or sets of tissue grafts with particular properties. Thus, the present invention provides solutions to the issues of inconsistency of properties between tissue grafts and the lack of an available source for tissue grafts with standardized properties. Particular pre-selected properties of the standardized tissue grafts include, for instance, amniotic membrane particle size, viscosity, and amniotic membrane concentration, which are standardized to enhance suitability of the tissue graft to a particular location in or on a subject. In addition, the invention also provides sets of tissue grafts with ranges of values for particular properties, for example, amniotic membrane particle size, that vary by no more than a pre-determined amount (e.g., a standardized tissue graft where the average amniotic membrane particle size in the tissue graft varies by no more than 10%).

Disclosed is a standardized tissue graft comprising a standardized quantity of dried amniotic membrane and a fluid, wherein the fluid rehydrates the dried amniotic membrane in a standardized concentration.

In some embodiments, the fluid is an isotonic electrolyte solution. In some embodiments, the fluid is a cryopreservative. In still other embodiments, the fluid is an isotonic electrolyte solution and a cryopreservative.

In some embodiments, the amniotic membrane is a mammalian amnion. In some embodiments, the amniotic membrane is a human amnion.

In some embodiments, the standardized concentration of dried amniotic membrane after rehydration is less than 0.5 mg/ml. In some embodiments, the standardized concentration of dried amniotic membrane after rehydration is between 0.5 mg/ml and 1.0 mg/ml. In some embodiments, the standardized concentration of dried amniotic membrane after rehydration is between 1.0 mg/ml and 1.5 mg/ml. In some embodiments, the standardized concentration of dried amniotic membrane after rehydration is between 1.5 mg/ml and 2.0 mg/ml. In some embodiments, the standardized concentration of dried amniotic membrane after rehydration is between 2.0 mg/ml and 2.5 mg/ml. In some embodiments, the concentration of dried amniotic membrane after rehydration is between 2.5 mg/ml and 3.0 mg/ml. In some embodiments, the concentration of dried amniotic membrane after rehydration is between 3.0 mg/ml and 3.5 mg/ml. In some embodiments, the concentration of dried amniotic membrane after rehydration is between 0.5 mg/ml and 1.5 mg/ml, 1.5 mg/ml and 2.5 mg/ml, or 2.5 mg/ml and 3.5 mg/ml. In some embodiments, the concentration of dried amniotic membrane after rehydration is between 3.5 mg/ml and 10 mg/ml. In some embodiments, the concentration of dried amniotic membrane after rehydration is between 10 mg/ml and 100 mg/ml. In some embodiments the concentration of dried amniotic membrane after rehydration is greater than 100 mg/ml.

In some embodiments, the invention includes a set of tissue grafts, wherein each tissue graft in the set comprises a standardized tissue graft that includes a standardized quantity of dried amniotic membrane and a fluid that rehydrates the dried amniotic membrane in a standardized concentration. For example, in various embodiments, the set of tissue grafts includes standardized tissue grafts in which the standardized concentration of dried amniotic membrane that has been rehydrated is less than 0.5 mg/ml, between 0.5 mg/ml and 1.0 mg/ml, between 1.0 mg/ml and 1.5 mg/ml, between 1.5 mg/ml and 2.0 mg/ml, between 2.0 mg/ml and 2.5 mg/ml, between 2.5 mg/ml and 3.0 mg/ml, between 3.0 mg/ml and 3.5 mg/ml, between 3.5 mg/ml and 10 mg/ml, between 10 mg/ml and 100 mg/ml, or greater than 100 mg/ml.

In some embodiments, the invention includes a set of standardized tissue grafts wherein each tissue graft in the set comprises a standardized tissue graft that includes a standardized quantity of dried amniotic membrane and a fluid that rehydrates the dried amniotic membrane in a standardized concentration that varies by no more than a known amount. For instance, in some embodiments for each tissue graft in the set of standardized tissue grafts, the concentration of dried amniotic membrane varies by no more than 1%, no more than 5%, no more than 7.5%, no more than 10%, no more than 15%, no more than 20%, nor more than 25%, no more than 30%, no more than 40%, or no more than 50%.

In some embodiments, the invention includes a standardized tissue graft that includes a standardized quantity of dried amniotic membrane and a fluid that rehydrates the dried amniotic membrane at a standardized concentration, where the average size of the amniotic membrane particles varies by no more than a known amount. For instance, in some embodiments the average amniotic membrane particle size of the standardized tissue graft varies by no more than 1%, no more than 5%, no more than 7.5%, no more than 10%, no more than 15%, no more than 20%, nor more than 25%, no more than 30%, no more than 40%, or no more than 50%.

Similarly, in some embodiments, the invention includes a set of standardized tissue grafts wherein each tissue graft in the set comprises a tissue graft or a standardized tissue graft that includes a standardized quantity of dried amniotic membrane and a fluid that rehydrates the dried amniotic membrane, where the average size of the amniotic membrane particles varies by no more than a known amount. In some embodiments, the invention includes a set of standardized tissue grafts wherein each tissue graft in the set comprises a tissue graft or a standardized tissue graft that includes a standardized quantity of dried amniotic membrane and a fluid that rehydrates the dried amniotic membrane at a standardized concentration, where the average size of the amniotic membrane particles varies by no more than a known amount. For instance, in some embodiments for each tissue graft in the set of standardized tissue grafts, the average amniotic membrane particle size varies by no more than 1%, no more than 5%, no more than 7.5%, no more than 10%, no more than 15%, no more than 20%, nor more than 25%, no more than 30%, no more than 40%, or no more than 50%.

In some embodiments, the invention comprises a set of standardized tissue grafts that includes a minimum number of standardized tissue grafts. For instance, a set of standardized tissue grafts of the invention may include at least 1, at least 5, at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, or at least 1000 standardized tissue grafts.

In some embodiments, the fluid that rehydrates the dried amniotic membrane of each tissue graft in a set of standardized tissue grafts at a standardized concentration, rehydrates the amniotic membrane at a concentration of less than 0.5 mg/ml, between 0.5 mg/ml and 1.0 mg/ml, between 1.0 mg/ml and 1.5 mg/ml, between 1.5 mg/ml and 2.0 mg/ml, between 2.0 mg/ml and 2.5 mg/ml, between 2.5 mg/ml and 3.0 mg/ml, between 3.0 mg/ml and 3.5 mg/ml, between 3.5 mg/ml and 5 mg/ml, between 3.5 mg/ml and 10 mg/ml, between 10 mg/ml and 100 mg/ml, or greater than 100 mg/ml. In some embodiments, the fluid rehydrates the dried amniotic membrane at a concentration of between 0.5 mg/ml and 5 mg/ml.

In some embodiments, the invention includes a standardized tissue graft comprising a dried amniotic membrane comprising a standardized particle size and a fluid that rehydrates the dried amniotic membrane at a standardized concentration. Similarly, in some embodiments, the invention includes a set of standardized tissue grafts wherein each tissue graft in the set comprises a tissue graft or a standardized tissue graft that includes a dried amniotic membrane comprising a standardized particle size and a fluid that rehydrates the dried amniotic membrane at a standardized concentration. In some embodiments, the standardized particle size is less than about 50 microns, between about 50 microns and 100 microns, between about 100 microns and 200 microns, between about 200 microns and 500 microns, between about 500 microns and 750 microns, between about 750 microns and 1 millimeter, or larger than 1 millimeter.

The foregoing and other features and advantages of the present invention will be apparent from the following more detailed description of the particular embodiments of the invention, as illustrated in the accompanying drawings.

### BRIEF DESCRIPTION OF THE DRAWINGS

FIG. 1 is a representation of ground amnion 110 suspended in fluid 100.

FIG. 2 is a schematic overview of steps resulting in the formation of a standardized amniotic membrane tissue graft.

### DETAILED DESCRIPTION OF EMBODIMENTS OF THE INVENTION

Fetal placental membranes (“PMs”) occupy a unique position in the field of regenerative medicine. This tissue, which derives solely from the developing embryo and fetus, comprises amnion (amniotic membrane or “AM”) and chorion (chorionic membrane or “CM”) fused at a basement-membrane/stroma interface and contains a dense concentration of extraembryonic mesenchymal stem cells (“SCs”) in an interstitial matrix rich with multiple classes of biologically active molecules.

The AM is a single layer of epithelial cells—amniocytes—on a thick basement membrane/connective tissue stroma. It derives from the embryonic epiblast, which is adjacent to the primitive streak and contiguous with cells giving rise to the notochord, and grows into a fluid-filled sac enveloping the developing fetus. Because of the amnion's embryonic origin at the earliest stage of development, amniocytes comprise pluripotent stem cells capable of engraftment and differentiation in host tissue of another individual. Additionally, amniocytes neither express HLA Class I antigens nor have immune-competence associated with hematopoietic stem cells. Consequently, transplanted amniocytes do not provoke an immune response in the recipient and do not differentiate into host-sensitized T-lymphocytes capable of mounting a graft-versus-host reaction. Amniocytes, in fact, are so immunoprivileged that cross-species engraftment of donor amniocytes as xenografts has been reported in otherwise immuno-competent animals.

The CM is a more complex tissue arising from the embryonic trophoblast. The chorion side of the placenta develops adjacent to and invades the maternal uterine wall, developing as the nutrient and gas exchange interface between maternal and fetal blood. In contrast to the histologically simple AM, the CM is considerably more complex, containing several distinct cell and tissue types. First, the trophoblast is a tissue on the uterine surface of the chorion and contains subpopulations of embryonic cells. The extravillous cytotrophoblast, a sub-population of the trophoblast, invades the maternal endometrium. Second, the syncytiotrophoblast forms a syncytium of densely nucleated cytoplasm covering the chorionic villi and directly contacting the maternal blood. Like the AM, the CM is also rich in undifferentiated extraembryonic mesenchymal stem cells. Unlike the AM, CM is used less extensively as a tissue graft because of its immunogenicity.

AM for tissue graft preparations is potentially available in substantial quantities. There are just under 4 million births per year in the United States, which make up the potential donor pool. From this pool, AM is made available from a suitable subpopulation of donors. Donors undergo a pre-donation screening process to minimize the risk of transmission of maternal or fetal infections agents by way of donated fetal membranes to an eventual tissue graft recipient. This screening procedure includes subjective and objective components. The subjective component includes screening by donor questionnaire for social high-risk behaviors for infectious disease. Some paid donors are motivated to hide a past social history of high-risk behavior for transmission of sexually transmitted infections, including hepatitis B virus (“HBV”), hepatitis C virus (“HCAV”), and human immunodeficiency virus (“HIV”). Accordingly, only volunteer donors are used. The objective (pre-delivery) screening component includes a metabolic panel including liver function studies and assessment of serology for evidence of past or present HBV, HCV, or HIV infection.

Placental membranes from acceptable donors may be excluded by perinatal observations and events. Clinical or laboratory evidence of active maternal or fetal infections around the time of delivery, the most sever example manifest by chorioamnionitis, precludes the use of fetal membranes for grave preparation. Meconium staining of the AF and/or the fetal membranes, although usually not indicative of infection, also eliminates the tissue from the donor pool. Finally, and most commonly, contamination of the placental membranes with a large quantity of maternal blood, feces, or other perinatal sources of gross bacterial or tissue contamination precludes use of the fetal membranes.

Deliveries of placental membranes follow delivery of the infant, and are vaginal or by way of a surgical Cesarean section (“Cesarean”). The use of AM from donors undergoing a Cesarean delivery largely eliminates gross bacterial contamination of the placental membranes during delivery. Consequently, AM is preferentially procured during a Cesarean, and not a vaginal, delivery. Of the approximately 4 million annual U.S. births mentioned earlier, approximately 33%—1.32 million overall—are by Cesarean delivery. This practice, therefore, reduces the potential donor pool by nearly seventy percent. Fetal placental membranes, including AM, may also be collected during a routine vaginal delivery, however proper precautions and additional processing steps are necessary. The bacterial contamination of PMs that occurs with vaginal delivery of the placenta is usually minimal in an uncomplicated delivery but still must be addressed. PMs used as tissue grafts collected from a vaginally delivered placenta may be effectively treated with sterile washings using topical antibiotic and non-tissue-toxic antimicrobial solutions immediately following delivery and thereafter. Therefore, AM is potentially available for use as a tissue graft from a total between 3.5 and 4.0 million births annually in the U.S.

Regardless, AM suitable for use as a tissue graft is not universally available through a Cesarean delivery. Gross contamination rendering the AM unsuitable for tissue grafting may occur during the delivery itself, or later during processing and/or packaging. Thus, retrieving useful AM from vaginal deliveries for preparation of tissue grafts and other applications is desirable.

As briefly mentioned, AM may be collected from suitable volunteer donors and processed for storage prior to use as a tissue graft in a variety of surgical procedures. AM is used in a plethora of surgical procedures and non-surgical applications. Some examples include use of AM as a biologic dressing/wound covering, a substrate foe the creation of artificial skin, and to promote healing of chronically ischemic or infected wounds. Surgical uses of AM tissue grafts include introduction as an adjunct to healing of surgically repaired bone, tendon, other soft tissue, and open wounds; a means to militate the formation of scar tissue and adhesions, and other beneficial applications in surgery and non-surgical minimally invasive medical therapies. AM and AM derivatives are used as biologic dressings containing a source of SCs and growth factors to treat burns, skin pressure ulcers, other chronic open wounds, corneal ulcers, and as a dressing following corneal transplant and other ocular procedures. AM tissue grafts are used to address soft tissue defects and facilitate healing following debridement and repair of damaged cartilage, tendon, bone, nerve, and muscle tissue. AM is under investigation as a connective tissue scaffolding for tissue and organogenesis using extraembryonic SCs and other progenitor cells. Fludized AM tissue grafts, possession the anti-inflammatory properties of AM, may be used to prevent the development of postoperative adhesions between the tendon, tendon sheath, and associated tissue following tenolysis, synoviolysis, surgical repair of a damaged tendon, and surgical debridement of necrotic or damaged tendon tissue. Fluidized AM tissue grafts are also useful to mitigate nerve cell death and promote axonal regeneration following early repair of peripheral nerve transections.

An injectable AM tissue graft preparation allows for expanded use of the product in both surgical and minimally invasive settings. The AM tissue graft may be injected into a defined closed space near the end of the surgical procedure, but prior to closing superficial layers of muscle, fascia, and skin at a time when precise placement of the tissue graft under the surgeon's direct visualization is possible. For example, an injectable AM tissue graft, depending on the viscosity of the final product, is delivered by injection though a hypodermic needle as small as 30-gauge (“G”) into a closed tendon sheath following tenolysis or tendon repair, into a closed joint capsule following repair of intra-articular cartilage, ligaments, or total joint replacement, into the peritoneal cavity following closure of the abdominal wall, into the pleural space following closure of the chest wall, and into the subdural space following closure of the spinal or intracranial dura mater. An injectable AM tissue graft of higher viscosity is injected through a 23G, 22G, 21G, 20G, 18G, 16G, or larger-bore hypodermic needle in these and other surgical and minimally invasive applications. An injectable AM tissue graft of lower viscosity is injected through a 25G or 30G needle for use in fine neural repair, aesthetic surgery, and other applications. Following wound closure, an injectable AM tissue graft may also be re-injected into the defined closed space during the perioperative and postoperative period if deemed useful by the surgeon or other healthcare provider.

An injectable AM tissue graft may also be injected into a tissue bed in a minimally invasive non-surgical setting. A syringe containing a quantity of AM tissue graft is fitted with a hypodermic needle of suitable size for the intended application. The needle is directed to the target tissue bed using visualization and palpation of external landmarks by the provider. Placement of the needle within the target tissue space or tissue may, in some embodiments, be facilitated with fluoroscopy or other non-invasive and minimally invasive imaging modalities. Some example uses of the injectable AM tissue graft include intra-articular injection for treatment of injured ligaments, cartilage, and bone; intra-capsular injection of tendon injuries, synovitis, tenosynovitis, and other inflammatory joint conditions; intra-thecal injection for treatment of spinal cord and brain injuries, aseptic meningitis, and other central neurological infections and inflammatory conditions; and other minimally invasive non-surgical applications.

In all of these and other applications, there is strong evidence that the presence active biomolecules in the AM-derived dressing or allograft improves healing across a broad range of tissue types, locations within the body, and applications. Reporting of clinical results may eventually lead to the use of AM and AM-derived preparations as a standard therapy and possibly even a best practice for the treatment of a variety of conditions. Such reporting requires continued laboratory experimentation and human clinical trials to generate additional data for review and interpretation in light of currently available practices and results therefrom. Meaningful interpretation of these data, however, depends on reproducibility. Reproducibility requires standardization of materials and techniques. Such standardization in this area should include the total tissue weight per volume and the concentration of small and large-molecule biologically active compounds present in the tissue graft used.

Preparation and sterilization of AM for later use as a tissue graft typically includes drying, packaging, sterilization, and storage. Drying discourages bacterial growth and helps maintain sterility during storage. Drying facilitates standardization of the final AM tissue graft in terms of weight per unit volume of dried AM prepared under standardized parameters of temperature, humidity, and time. Drying may be accomplished by heating or freezing under controlled conditions to minimize water-ice crystal formation and cellular disruption in products wherein preservation of SCs is desired. Although some viable SCs are preserved by drying and/or freezing under controlled conditions, other SC's die during processing. Additionally, it is believed that tissue grinding completely disrupts the cellular elements. It is not fully known how drying and storage affect the concentration of the biologically active non-cellular components of AM, though a significant decrease in concentration of intact proteins and other large biomolecules is possible. Sterilization by heat or radiation destroys the cellular components of AM preparations, including SCs. Thermal or irradiative sterilization methods may also denature proteins and alter or destroy other large biologically active molecules. Some graft preparations reconstitute the dried AM using a tissue preservative solution prior to packaging and storage.

It is useful to employ AM tissue graft preparations of varying viscosity for transplantation with knowledge of expected results based upon reproducibility. Variations in viscosity affect the tendency of the AM tissue graft to remain and engraft at the site of placement. Differences in viscosity are considered based upon the intended use of the standardized AM tissue graft. Generally, standardized AM tissue grafts are prepared in three reproducible, standardized viscosities: high viscosity; medium viscosity; and low viscosity.

High-viscosity standardized AM tissue graft has a concentration of ground AM of greater than 10 mg/ml, with or without an additional biologically compatible “thickening agent.” Some examples of applications where a high-viscosity standardized AM tissue graft may be used include the non-invasive or minimally-invasive treatment of entero-cutaneous, entero-vaginal, entero-enteric, broncho-pleural, tracheal-esophageal fistulas; graft-repair of osteochondral defects in the knee, hop, ankle, wrist, hand, and other joints; microfractures and small facial fractures; and filling of large bone tissue void following surgical treatment of certain cancers.

Medium-viscosity standardized AM tissue graft has a concentration of ground AM of between 1 mg/ml and 10 mg/ml. Examples of applications where a medium-viscosity standardized AM tissue graft may be used include treatment of wound sinus tracts, grafting of cutaneous and soft-tissue defects resulting from deep thermal or radiation burns; spinal and other bony fusion procedures (when combined with currently available bone putty or as a stand-alone application into a cervical or lumbar intervertebral spacer); facial trauma and facial fracture treatment; bone grafting; alveolar cleft (“cleft palate”) grafting; treatment of dental/tooth tissue defects; chronic inflammatory bursitis; intervertebral facet-based pain; tears of the meniscal cartilage; application to entero-entero and other surgical anastomoses; treatment of non-union and mal-union of fractures, intra-peritoneal application following surgical adhesiolysis; intra-peritenon implantation following Achilles' tendon debridement and anastamotic repair; defects of the calvarium following trauma; emergency decompressive craniotomy; surgical breast reconstruction; and following acetabular and other articular joint surface resurfacing.

Low-viscosity standardized AM tissue graft has a concentration of ground AM of less than 1 mg/ml. Examples of applications where a low-viscosity standardized AM tissue graft may be used include treatment of chronic wounds, radiation burns, and thermal injury by subcutaneous injection; injection into peri-rotator cuff soft tissues following rotator cuff repair; injection to facilitate non-surgical repair and healing of supraspinatus, infraspinatus, teres minor, and subscapularis tears; other muscle, ligament, tendon, and soft-tissue tears; epicondylitis; and other similarly debilitating chronic fascial inflammatory conditions such as plantar fasciitis or fasciolosis.

Substantial differences in both the absolute amount and concentration per unit volume of biologically active substances in the final preparation arise in currently available preparations based upon the preparation methods used. Existing AM tissue graft preparations are typically formed by suspending a single ground amnion in a suitable fluid. The weight of an individual amnion, however, is variable. Although recording placental weights may not directly reflect amnion weights, a published study (Lurie, et al. (1999) “Human fetal-placental weight ratio in normal singleton near-term pregnancies” Gynecologic and Obstetric Investigation, 48(3): 155-57.) of 431 uncomplicated singleton deliveries revealed a mean placental weight of 613 +/−123.8 mg, ranging from 319 mg to 1,266 mg. Thus, the weight of the human placenta and its constituent components commonly ranges by nearly 40% around the mean weight and may vary by as much as 400%. The commonly used techniques in preparation of AM-derived suspensions, therefore, result in a preparation with a completely arbitrary total amount and concentration of AM. In some preparation methods, AM is subject to different degrees of drying, whether intentionally or unintentionally. Random samples of AM processed and stored in non-standardized conditions with respect to temperature and drying time revealed an average weight of 1.02 +/−0.12 mg/cm².

What is lacking in the prior art, therefore, is an AM-derived tissue graft preparation incorporating an effective concentration of products of SC disruption and active biomolecules from an individual donor or the largest possible pool of volunteer donors in a standardized, reproducible concentration.

Embodiments of this invention address these and other fundamental AM tissue graft requirements—high concentration of beneficial biomolecules in a standardized preparation with no antigenic material and minimal waste of available donor tissue—by forming a tissue graft comprising a preparation of dried particulate AM rehydrated by an acceptable fluid in standardized quantities and concentrations.

Disclosed is a standardized tissue graft preparation comprising dried and ground amniotic membrane reconstituted with an acceptable fluid in known concentrations. The preparation is used by medical providers as an injectable fluid or non-injectable gel combination tissue graft, either by intraoperative application or injection, non-operative percutaneous injection, or direct application to injured, ischemic, infected, or otherwise damaged tissue. The preparation is also used by laboratory researchers as a reproducible source of standardized material for basic science research of the effects of AM preparations on healthy, diseased, and damaged tissue in the field of regenerative medicine, orthopedics, neurology, neurosurgery, gynecologic surgery, and in other clinical, basic medical science, and related scientific disciplines. The use of standardized quantity of ground, dried AM reconstituted in a suitable fluid such as an isotonically balanced buffered electrolyte solution and/or cryopreservative, maximizes delivery of a wide range of beneficial biologic substances within a non-antigenic liquid or gel tissue graft to the treatment site.

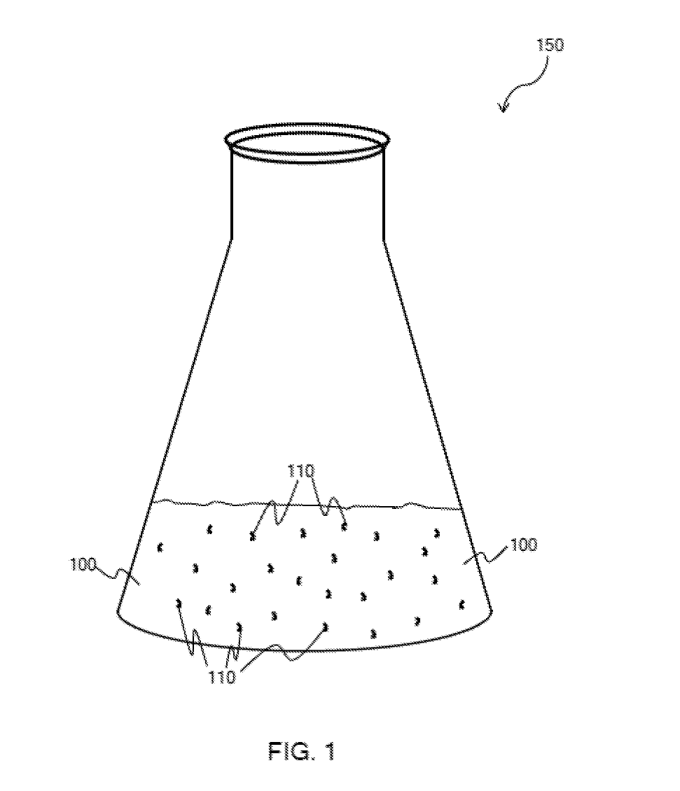

FIG. 1 shows a standardized AM tissue graft 150 comprising a ground AM 110 suspended in a fluid 100. Details regarding the composition and preparation of the fluidized AM tissue graft are provided below and throughout this disclosure.

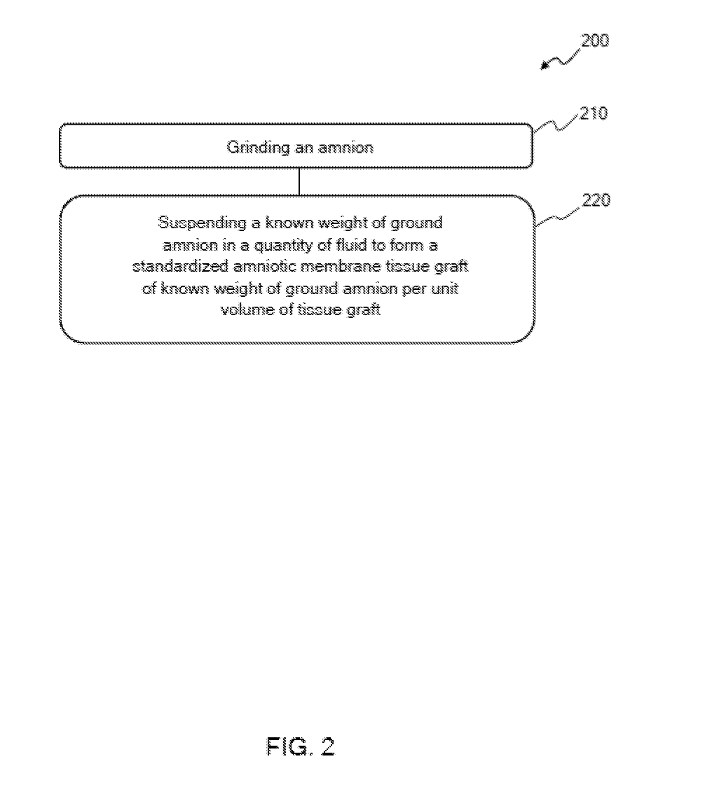

FIG. 2 shows a schematic overview 200 of the processing steps utilized through some embodiments of the invention to create a standardized AM tissue graft. Overview 200 requires an amnion. In some embodiments of the invention, the AM is received from a volunteer human donor. Accepting PMs from volunteer donors and excluding non-volunteer and/or paid donors from the donor pool is consistent with internationally swell-established tissue donation protocols because it reduces the chance that an infectious agent present in the donor will be transmitted to the graft recipient, resulting in and infection in the recipient. Screening of potential volunteer donors, therefore, includes obtaining a comprehensive past medical and social history, complete blood count, liver and metabolic profile, and serologic testing for HBV, HCV, HIV, and other infectious agents, in some embodiments.

Non-human AM donors are used in some embodiments of the invention. A lack of expression of HLA-1 and HLA-DR epitopes makes cross-species transplantation of AM possible. A non-human AM which is de-cellularized by enzymatic or chemical treatment prior to tissue grinding may remove additional possible antigens.

In some embodiments, donor tissue is obtained during delivery by elective Cesarean section. The use of a Cesarean-delivered AM to prepare the standardized AM tissue graft is preferable in some embodiments because an AM delivered by Cesarean section is obtained and packaged under strict sterile technique in the operating room, with essentially no/minimal microbial contamination. Following Cesarean delivery of the infant, the placenta is delivered. Operating room personnel familiar with sterile technique and tissue handling perform all steps necessary to prepare the tissue for packaging. The combined fetal placental membranes (AM and CM) are dissected from the maternal placental plate (decidua). The combined fetal placental membranes are gently washed with sterile 0.9% saline solution to remove all visible traces of maternal blood, AF, and any other visible, potentially contaminating material. The dissected and washed combined fetal placental membranes are then placed in a sterile specimen container and a quantity of 0.9% sterile saline is added sufficient to completely submerge the combined fetal placental membranes. The sterile container containing the fetal placental membranes collected under sterile conditions in the operating room are then securely closed and placed in a donor tissue specimen bag. This first bag is then placed within a second bag, which is then sealed, labeled, and taken from the operating room for packaging in an insulated ice-bath container. A patient data sheet containing information regarding the maternal donor is placed in the container, and a separate copy of this information is recorded and logged prior to closing the package. The packaged specimen container is then immediately transported to the processing facility by staff who rotate on call, such that there is minimal delay following delivery before the donor tissue arrives at the separate facility for processing.

Despite the preference for a Cesarean-delivered AM, vaginally delivered fetal membranes are utilized in some embodiments to increase the pool of potential donors and for other of the aforementioned reasons. Great care must be afforded the vaginally-delivered placental tissue to prevent microbial contamination. Vaginally-delivered fetal membranes are not acceptable donor tissue if there is fecal or other grossly visible contamination, or if there is substantial contact of the placental membranes with clothing, bedding, non-sterile unprepped skin, or other non-sterile surfaces during delivery or prior to sterile packaging. Neither a vaginally-delivered AM nor a Cesarean-delivered AM is acceptable donor tissue if there is visible staining of the fetal membranes with meconium. Following delivery, the steps for preparing vaginally delivered fetal membranes are the same as the above description of preparing Cesarean-delivered fetal membranes. A fully gowned-and-gloved staff member processes the fetal membranes on a sterile field established on a back table, or similar surface, in the labor/delivery room. An additional step comprising rinsing the vaginally delivered fetal membranes with an antimicrobial solution is used in some embodiments. After washing with 0.9% sterile saline, the vaginally delivered dissected fetal membranes are washed with a topical antimicrobial solution. Examples of the topical antimicrobial solution used to wash the vaginally delivered fetal membranes, in some embodiments, are a 0.5% aqueous solution of glutaraldehyde (which is then washed off the donor tissue using a final rinse of 0.9% sterile saline prior to packaging), a penicillin-streptomycin solution comprising 50-100 International Units (“IU”) per ml of penicillin and 50-100 micromg/ml of streptomycin, or a 0.0125% aqueous solution of sodium hypochlorite. These examples are not meant to be limiting. Other antimicrobial solutions toxic to infectious microorganisms at non-cytotoxic concentrations may also be used. The fetal membranes, following the antimicrobial washing, are then placed in a sterile specimen container, covered with 0.9% sterile saline solution, and sealed in sequential sterile bags as described above for Cesarean-delivered fetal membranes. The prepared, sealed, labeled, recorded, and packaged donor fetal membranes are then delivered to the separate tissue processing facility, as described above.

Immediately upon receipt at the processing facility, the shipping label is examined and information regarding the specimen and donor is recorded. The shipping container is examined for integrity, including confirmation of an intact tamper-proof seal. The shipping container is then opened and the inner bag containing the placental membranes is examined. An infrared temperature sensor is directed at the tissue bag to confirm a temperature of between 6 and 10 degrees Celsius. If there is any indication of damage to the outer container, the inner bag containing the placental membranes is examined with particular care. If damage to the inner bag is identified or the tamper-proof seal is broken or damaged, the specimen is not used to prepare the tissue graft. A donor/specimen data sheet within the container is then reviewed to validate the donor's credentials. The information on the data sheet is compared to the donor ID on the specimen bag to confirm the data sheet for the donor matches the specimen. This information is recorded and included in the permanent batch record for that specific donor. These credentials include donor lot numbers and expiration dates. All validation dates and times are confirmed. A donor tissue specimen that is unacceptable for any reason is discarded. The date, time, and hospital from which the donor specimen was received are recorded. The outside of the bag containing the two separate sterile specimen containers is then sprayed with isopropyl alcohol and manually wiped down. The logged and cleaned specimen bag containing the donor placental membranes is then stored in a locked refrigerator in an ice water bath, but not frozen.

Following receipt of the donor tissue, the amnion is cleaned and prepared for grinding, as practiced in some embodiments shown in FIG. 1. Under strict sterile technique, the specimen bag is opened using sterile scissors and the donor specimen comprising placental membranes is carefully poured into a large sterile basin. Using sterile forceps, the AM is peeled from the CM, which separates at the AM basement membrane/CM stromal interface. The AM is placed on a sterile cutting board, CM-side facing up. The CM side is gently wiped with sterile cloth towels, taking care to remove any adherent bits of CM and clotted blood which may not have been completely rinsed from the AM immediately following the delivery prior to packaging. Both sides of the AM are once again washed with sterile 0.9% saline and rinsed with an antimicrobial solution in some embodiments, such as 0.5% aqueous solution of glutaraldehyde for example.

In some embodiments, a dried or partially dried AM is used for grinding. In some embodiments of the invention, the AM is ground fresh and not dried prior to grinding. Using sterile scissors, the cleaned and treated processed AM is cut into generally rectangular sections measuring approximately three inches wide and eight inches long. This is by way of example, and in no way meant to be limiting. In embodiments where a dried or partially dried AM is used for grinding, the cut sections of AM are placed on a sterile pan and partially dried at ambient temperature.

As shown in FIG. 2 the first step 110 of overview 200 is grinding an amnion. In some embodiments, for example, the AM is placed in a temperature-controlled ball-grinding mill (i.e. “CryoMill” for cryogenic grinding, manufactured by Retsch Corporation, Haan, Germany). In some embodiments, to prepare the final standardized tissue graft, the grinding jar and grinding balls are weighed prior to placement of a quantity of AM in the grinding jar. AM is then placed in the grinding jar. The AM, in various embodiments, may be fresh, dried, or partially dried. Because, in some embodiments, the ground AM will be substantially driede as a result of the grinding process, the ground, dried AM will be, essentially, completely dehydrated regardless of whether a hydrated, fresh AM or a partially dried AM was placed in the tissue mill. After placement in the grinding jar, the AM is pre-cooled in a liquid nitrogen bath to minus 196° Celsius and then ground for approximately 4 minutes. This process results in an AM particle size of 5 microns. This is by way of example not meant to be limiting in any way. Longer grinding times result in an AM particle size smaller than 5 microns and shorter grinding times result in an AM particle size of larger than 5 microns. The grinding jar is again weighed, and the weight of dehydrated, ground AM 110 contained within is determined. Determination of the weight of ground AM 110 allows for standardization of the AM tissue graft 150, as provided in some embodiments of the invention. These examples are not meant to be limiting. The grinding process may be longer or shorter than 4 minutes and the ground AM 410 particle size may be larger or smaller than 5 microns, depending on the desired viscosity, final concentration of AM/unit volume in the tissue graft, and other factors desired by the surgeon or other end-user health care provider.

Also shown in FIG. 2, step 120 of overview 200, in some embodiments, comprises suspending the ground AM 110 in a quantity of fluid 100 to form a standardized AM tissue graft. In some embodiments, the grinding jar containing the gound particulate AM 110 is opened and the ground AM 110 is washed from the jar and balls using a measured quantity, usually 50 cc's or less for example, of a fluid 100. In some embodiments, fluid 100 is a buffered isotonic solution (an example is “Plasma-Lyte A,” manufactured by Baxter International, Inc., Deerfield, Ill.). In some embodiments, fluid 100 is a cryoprotectant (an example is CryoStor CS-10, a 10% solution of dimethylsulfoxide (“DMSO”), manufactured by BioLife Solutions, Inc., Bothel, Wash.). These examples are not meant to be limiting, other examples of non-cytotoxic fluids may be used. In some embodiments, the grinding jar is weighed prior to opening. In some embodiments, a standard quantity of fluid 100, 50 cc's for example, is added to liquefy and reconstitute the ground AM 110. In some embodiments, the reconstituted AM (“AMFL”) has a known weight of AM per volume of AMFL. The formed standardized AM tissue graft 150 comprises the reconstituted AMFL.

Step 160, in some embodiments, comprises suspending the ground AM 410 in a quantity of fluid 400 to form an injectable AM tissue graft with a predetermined, standardized concentration of AM by weight. In some embodiments, the grinding jar containing the ground particulate AM is opened and the ground AM 410 is washed from the jar and balls using a measured quantity, usually 50 cc's or less for example, of a fluid 400. In some embodiments, the fluid 400 is a buffered isotonic solution (an example is “Plasma-Lyte A,” manufactured by Baxter International, Inc., Deerfield, Ill.). In some embodiments, the fluid 400 is a cryoprotectant (an example is CryoStor CS-10, a 10% solution of dimethylsulfoxide (“DMSO”), manufactured by BioLife Solutions, Inc., Bothel, Wash.). In some examples, the fluid is any combination of a buffered isotonic solution, a cryoprotectant, and any other suitable fluid. These examples are not meant to be limiting, other examples of non-cytotoxic fluids may be used, alone or in combination. In some embodiments, the grinding jar is weighed prior to opening. In some embodiments, a standard quantity of fluid 400, 50 cc's for example, is added to liquefy and reconstitute the ground AM 410. In some embodiments, the reconstituted AM (“AMF”) has a known weight of AM per volume of AMF. The standardized AM tissue graft 450 comprises the reconstituted AMF.

Additional quantities of AMF, ground AM by weight, a cryo-protectant, buffered isotonic solution, or other suitable fluid may be further combined, in some embodiments, to form completed standardized AM tissue graft 150. Sterile materials are used and sterile technique is maintained. The previously recorded weight per volume of AM is noted such that the completed tissue graft is a known, standardized, reproducible product. Once the final dilution ratios have been confirmed and prepared, the measured individual components are poured into a large beaker and gently suspended by gently swirling the beaker and/or stirring with a glass rod or other suitable instrument.

In some embodiments, a small quantity of standardized tissue graft is drawn into a sterile 2 cc syringe and extruded through a 25 gauge needle to ensure the graft is sufficiently fluid to be percutaneously or intraoperatively injected into the recipient tissue bed. In some embodiments, the viscosity of the graft is adjusted by mixing an additional measured quantity of buffered isotonic solution with the graft, and recording the final concentration of AM per ml and SC per ml accordingly. In some embodiments, the final concentration of AM and/or SC per ml is adjusted with additional buffered isotonic solution to an end-user's pre-ordered concentration requirement, based upon the intended use of the completed tissue graft.

In some embodiments, step 220 also comprises determining the quantity of finished standardized AM tissue graft 150 requested by end user based upon the intended use of the completed AM tissue graft 150. In some embodiments, completed standardized tissue graft 150 is packaged according to standard ordered AM concentrations and total AM tissue graft 150 volumes. In some embodiments, AM tissue graft 150 is packaged in standard differing AM concentrations and viscosities based upon the mode used for delivery (injection versus intraoperative application, recipient host tissue type, other specific requirements, for example) and intended therapeutic use.

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above, and are intended to fall within the scope of the appended claims.

What is claimed is:

1. A standardized tissue graft comprising:
a standardized quantity of dried amniotic membrane; and
a fluid, wherein the fluid rehydrates the dried amniotic membrane in a standardized concentration.

2. The tissue graft of claim 1, wherein the fluid is a fluid selected from the group of fluids consisting of an isotonic electrolyte solution and a cryopreservative.

3. The tissue graft of claim 1, wherein the fluid comprises an isotonic electrolyte solution and a cryopreservative.

4. The tissue graft of claim 1, wherein the amniotic membrane is a human amnion.

5. The tissue graft of claim 1, wherein the standardized concentration of dried amniotic membrane is less than 0.5 mg/ml.

6. The tissue graft of claim 1, wherein the standardized concentration of dried amniotic membrane after rehydration is between 0.5 mg/ml and 1.5 mg/ml. The tissue graft of claim 1, wherein the standardized concentration of dried amniotic membrane after rehydration is between 1.5 mg/ml and 2.5 mg/ml.

8. The tissue graft of claim 1, wherein the standardized concentration of dried amniotic membrane after rehydration is between 2.5 mg/ml and 3.5 mg/ml.

9. The tissue graft of claim 1, wherein the standardized concentration of dried amniotic membrane after rehydration is between 3.5 and 10 mg/ml.

10. The tissue graft of claim 1, wherein the standardized concentration of dried amniotic membrane after rehydration is between 10 and 100 mg/ml.

11. The tissue graft of claim 1, wherein the standardized concentration of dried amniotic membrane after rehydration is greater than 100 mg/ml.

12. A set of standardized tissue grafts, wherein each tissue graft in the set comprises:
a standardized quantity of dried amniotic membrane; and
a fluid, wherein the fluid rehydrates the dried amniotic membrane in a standardized concentration.

13. The set of tissue grafts of claim 12, wherein for each tissue graft in the set the standardized concentration of dried amniotic membrane varies by no more than 10%.

14. The set of standardized tissue grafts of claim 12, wherein the set comprises at least 50 standardized tissue grafts.

15. The set of standardized tissue grafts of claim 12, wherein the set comprises at least 10 standardized tissue grafts.

16. A set of standardized tissue grafts, wherein each tissue graft in the set comprises:
a standardized quantity of dried amniotic membrane; and
a fluid, wherein the fluid rehydrates the dried amniotic membrane in a standardized concentration, and the average amniotic membrane particle size varies by no more than 25% between each standardized tissue graft in the set.

17. The set of standardized tissue grafts according to claim 16, wherein for each tissue graft in the set the average amniotic membrane particle size varies by no more than 10%.

18. The set of standardized tissue grafts of claim 16, wherein the set comprises at least 10 standardized tissue grafts.

19. The set of standardized tissue grafts of claim 16, wherein the set comprises at least 50 standardized tissue grafts.

20. The set of standardized tissue grafts of claim 16, wherein the standardized concentration is between 0.5 mg/ml and 5.0 mg/ml.

21. A standardized tissue graft comprising:
a dried amniotic membrane comprising a standardized particle size; and
a fluid, wherein the fluid rehydrates the dried amniotic membrane in a standardized concentration.

22. The standardized tissue graft of claim 21, wherein the standardized particle size is less than about 50 microns.

23. The standardized tissue graft of claim 21, wherein the standardized particle size is between about 50 microns and about 100 microns.

24. The standardized tissue graft of claim 21, wherein the standardized particle size is between about 100 microns and about 200 microns.

25. The standardized tissue graft of claim 21, wherein the standardized particle size is between about 200 microns and about 500 microns.

26. The standardized tissue graft of claim 21, wherein the standardized particle size is between about 500 microns and about 750 microns.

27. The standardized tissue graft of claim 21, wherein the standardized particle size is between about 750 microns and about 1 millimeter.

28. The standardized tissue graft of claim 21, wherein the standardized particle size is larger than about 1 millimeter.
